(19) United States
(12) Patent Application Publication
(10) Pub. No.: US 2025/0205148 A1
(43) Pub. Date: Jun. 26, 2025

(54) SLOW RELEASE PHARMACEUTICAL COMPOSITION MADE OF MICROPARTICLES

(71) Applicant: Debiopharm International SA, Lausanne (CH)

(72) Inventors: Bertrand DUCREY, Martigny (CH); Patrick GARROUSTE, Saxon (CH); Catherine CURDY, Allschwil (CH); Marie-Anne BARDET, Maracon (CH); Herve PORCHET, Cugy (CH); Eija LUNDSTROM, Lutry (CH); Frederic HEIMGARTNER, Marin-Epagnier (CH)

(21) Appl. No.: 18/967,155

(22) Filed: Dec. 3, 2024

Related U.S. Application Data
(63) Continuation of application No. 17/932,621, filed on Sep. 15, 2022, now abandoned.
Application No. 17/932,621 is a continuation of application No. 16/192,674, filed on Nov. 15, 2018, now abandoned.
Application No. 16/192,674 is a division of application No. 12/601,649, filed on Apr. 20, 2010, now Pat. No. 10,166,181.

(30) Foreign Application Priority Data
Jun. 6, 2007 (EP) 07109767.9
Oct. 27, 2007 (WO) PCT/IB2007/054372

(51) Int. Cl. A61K 9/00; A61K 9/16; A61K 9/19

(52) U.S. Cl. CPC A61K 9/0019; A61K 9/1647; A61K 9/19

(57) ABSTRACT

Pharmaceutical composition made of microparticles for the slow release of an active substance at least during a period covering the 6th month after injection of said composition, said composition comprising a group of microparticles made of a copolymer of the PLGA type which incorporate an active substance in the form of a water insoluble peptide salt; said copolymer furthermore comprising at least 75% of lactic acid and an inherent viscosity between 0.1 and 0.9 dl/g, as measured in chloroform at 25° C. and at a polymer concentration of 0.5 g/dL; said microparticles furthermore having a size distribution defined as follows: D (v,0.1) is between 10 and 30 micrometers, D (v,0.5) is between 30 and 70 micrometers, D (v,0.9) is between 50 and 1 10 micrometers.

### FIELD OF INVENTION

The invention relates to pharmaceutical compositions made of microparticles which are used for the slow release of an active substance.

It more precisely relates to such compositions which comprise a copolymer of lactic and glycolic acid (PLGA) incorporating, as active substance, a water insoluble peptide salt.

### STATE OF THE ART

Compositions as defined above are disclosed in Swiss patent CH 679 207 A5.

### DEFINITIONS

In the present text, the term “microparticle” has to be understood as a solid object of any shape, e.g. microsphere or microgranule, having a median diameter of less than 250 micrometers.

The expression D (v,0.5), also mentioned as “median diameter”, means that 50% of the microparticles have a diameter which is less than the indicated value. Hence, if D (v,0.5)=55 micrometers, 50% of the microparticles have a diameter which is less than 55 micrometers.

In the same way, D (v,0.1) means that 10% of the microparticles have a diameter which is less than the indicated value and D (v,0.9) means that 90% of the microparticles have a diameter which is less than the indicated value.

All above values are measured by laser diffraction.

The term “microgranule” refers to an object which is the result of a milling operation on an elongated product such as an extrudate.

The term PLGA XX/YY refers to a poly(D,L lactide-co-glycolide), where XX represents the lactide content, and YY represents the glycolide content. The ratio lactide/glycolide being expressed in mol percent.

The term “month” refers to 28 days.

### GENERAL DESCRIPTION OF THE INVENTION

The objective of the invention is to offer several improvements with respect to the state of the art.

One of those improvements is to provide a continuous and efficient slow release of the active substance during at least a period covering the 6^{th} month after injection of the composition.

To this effect the invention concerns a pharmaceutical composition made of microparticles for the slow release of an active substance at least during a period covering the 6^{th} month after injection of said composition, said composition comprising a group of microparticles made of a copolymer of the PLGA type which incorporate an active substance in the form of a water insoluble peptide salt; said copolymer furthermore comprising at least 75% of lactic acid and an inherent viscosity between 0.1 and 0.9 dl/g, as measured in chloroform at 25° C. and at a polymer concentration of 0.5 g/dL; said microparticles furthermore having a size distribution defined as follows:
D (v,0.1) is between 10 and 30 micrometers,
D (v,0.5) is between 30 and 70 micrometers,
D (v,0.9) is between 50 and 110 micrometers.

In a first embodiment, the composition consists of one single group of microparticles. In that case the lactide content of the PLGA is of at least 85% and the inherent viscosity is between 0.1 and 0.4 dl/g.

A release during at least 6 months can be obtained with the composition of this first embodiment.

The microparticles may be microspheres or microgranules.

In a second embodiment, the composition comprises a group of microparticles wherein the lactide content of the PLGA is of at least 85% and the inherent viscosity is preferably between 0.5 and 0.9 dl/g and more preferably between 0.63 and 0.67 dl/g.

The microparticles may be microspheres or microgranules

The group of microparticles defined in the second embodiment may advantageously be used for providing a slow and significant release during at least a period starting from the 4^{th} month after injection of the composition until and including the 6^{th} month.

In a third embodiment, the pharmaceutical composition furthermore comprises another group of microparticles made of a copolymer of the PLGA type having a lactide content between 70% and 80% which incorporates said active substance.

Both groups of microparticles may be present in a dose ratio (expressed in peptide content) close to 50:50.

The inherent viscosity of each group is between 0.5 and 0.9 dl/g.

Preferably the inherent viscosity of the other group is between 0.60 and 0.70 dl/g and in particular 0.65 dl/g and the inherent viscosity of the first group is the same as the one defined in the second embodiment.

The microparticles of the other group may be microgranules or microspheres.

Advantageously both groups of microparticles are microgranules.

One group of microparticules is advantageously obtained by mixing, in a solvent-free process, said PLGA with said water insoluble peptide salt.

When two groups of microparticles are present, one group may be used to provide a slow and significant release of the water insoluble peptide salt during at least the first three months after injection of the composition while the other group is used for a release starting from the 4^{th} month.

In a preferred embodiment the active substance is a LHRH agonist triptorelin (used as water insoluble salt such as the pamoate salt thereof) which may be efficiently used in the treatment of prostate cancer.

The LHRH agonist triptorelin is released in an important immediate amount within hours following injection and then shows a constant and significant release over a long period of at least 168 days, i.e. 6 months

### DETAILED DESCRIPTION OF THE INVENTION

The invention is discussed below in a more detailed way with examples, the first being illustrated by the following figure:

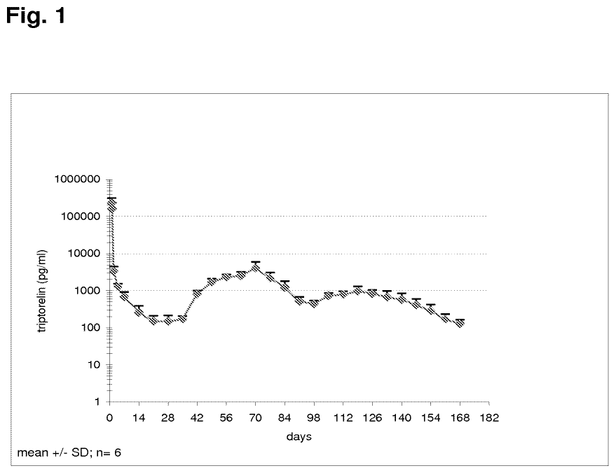

FIG. 1 shows triptorelin, LHRH agonist, serum levels obtained with the pharmaceutical biodegradable composition of example 1,

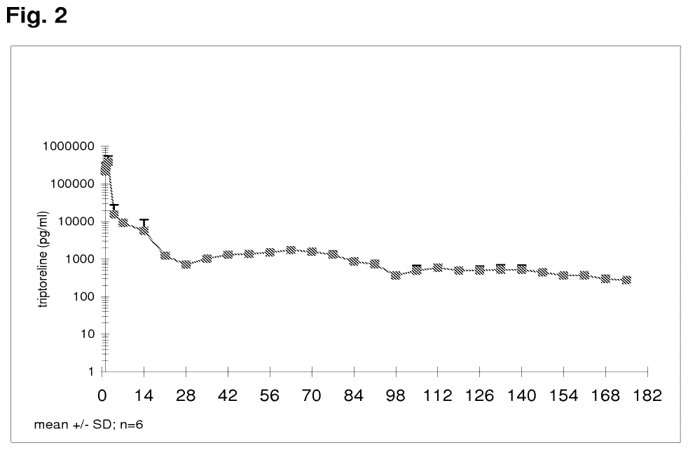

FIG. 2 shows triptorelin, LHRH agonist, serum levels obtained with the pharmaceutical biodegradable composition of example 3,

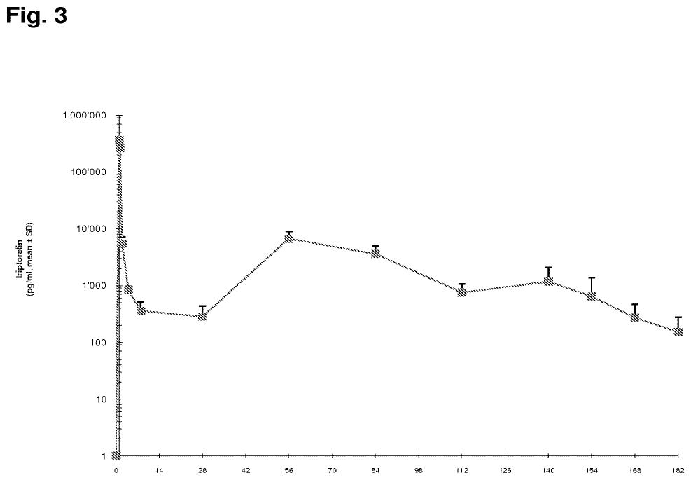

FIG. 3 shows triptorelin, LHRH agonist, serum levels obtained with the pharmaceutical biodegradable composition of example 4,

In the following examples the viscosity is expressed in dl/g and is measured at a polymer concentration of 0.5 g/dl.

### Example 1

A formulation of microgranules of triptorelin pamoate is prepared with the following process.

Approximately 12% (w/w) of triptorelin pamoate is mixed with approximately 88% (w/w) PLGA 75/25 having a viscosity of 0.65 dl/g, at room temperature. The given mixture is duly homogenized, subjected to progressive compression and simultaneously to a progressive heating, before extrusion. The extrudate is cut into pellets and ground at a temperature of about −100° C. The microgranules obtained after grinding are sieved below 180 micrometers. Their size distribution is defined as follows:
D (v,0.1)=23 micrometers
D (v,0.5)=55 micrometers
D (v,0.9)=99 micrometers

A formulation of microspheres of triptorelin pamoate and PLGA 85/15 having an inherent viscosity of 0.68 dl/g is prepared as follows:

Aqueous phase is prepared by mixing, under magnetic stirring, at a temperature of 40° C., 240 g of polyvinyl alcohol and 11760 g of purified water. In parallel, the organic phase is prepared by total dissolution of 12 g of polymer 85/15 poly(D,L lactide-co-glycolide) (PLGA) in 45 g of ethyl acetate under magnetic stirring.

3000 mg of triptorelin pamoate are suspended in 30 g of ethyl acetate and placed under magnetic stirring. This solution is incorporated to the organic phase previously prepared. The organic phase is then introduced in a homogenisation chamber simultaneously with the said aqueous phase. Both phases are mixed in order to obtain an emulsion and the extraction of the solvent from the organic phase and to isolate a suspension of microspheres.

Finally the formulation of microspheres is recovered by filtration and dried by lyophilization.

The microspheres have a size distribution defined as follows:
D (v,0.1)=15.6 micrometers
D (v,0.5)=33.4 micrometers
D (v,0.9)=60.8 micrometers

The formulation of microspheres and the formulation of microgranules are mixed in a vial in order to have a 50:50 dose ratio of each formulation. The mixture is suspended in an appropriate aqueous medium, lyophilised and sterilized by gamma irradiation.

The purity measured on the obtained pharmaceutical biodegradable composition is 98.3% and the burst evaluated in vitro (in a phosphate buffer pH 7.4) over a 6 hours period is 22.1%.

In this example, the obtained pharmaceutical formulation is tested in vivo and the animal model is the rat. The formulation as described above is suspended in water for injection and is administered at a concentration dose of 18 mg/kg to 6 rats.

The LHRH agonist triptorelin of said pharmaceutical biodegradable composition is released in an important immediate amount within hours following injection and then shows a constant and significant release over a long period of at least 168 days, i.e. 6 months.

### Example 2

A formulation of microgranules of triptorelin pamoate is prepared as described in example 1.

A formulation of microspheres of triptorelin pamoate is prepared as described in example 1 with PLGA 90/10 having an inherent viscosity of 0.7 dl/g. The microspheres have a size distribution defined as follows:
D (v,0.1)=17.6 micrometers
D (v,0.5)=39.9 micrometers
D (v,0.9)=84.2 micrometers

The formulation of microspheres and the formulation of microgranules are mixed in a vial in order to have a 50:50 dose ratio of each formulation. The mixture is suspended in an appropriate aqueous medium, lyophilised and sterilized by gamma irradiation.

The purity measured on the obtained pharmaceutical biodegradable composition is 98.3% and the burst evaluated in vitro (in a phosphate buffer pH 7.4) over a 6 hours period is 19.4%.

The LHRH agonist triptorelin of said pharmaceutical biodegradable composition is released in an important immediate amount within hours following injection and then shows a constant and significant release over a long period of at least 168 days, i.e. 6 months.

### Example 3

A formulation of microgranules of triptorelin pamoate is prepared as described in example 1.

Another formulation of microgranules is prepared as described in example 1 with PLGA 85/15 having an inherent viscosity of 0.66 dl/g.

Approximately 20% (w/w) of triptorelin pamoate is mixed with approximately 80% (w/w) PLGA 85/15 at room temperature. The given mixture is duly homogenized, subjected to progressive compression and simultaneously to a progressive heating, before extrusion. The extrudate is cut into pellets and ground at a temperature of about −100° C. The microgranules obtained after grinding are sieved below 180 micrometers. Their size distribution is defined as follows:
D (v,0.1)=20.5 micrometers
D (v,0.5)=51.7 micrometers
D (v,0.9)=96.9 micrometers

The 2 formulations of microgranules are mixed in a vial in order to have a 50:50 dose ratio of each formulation. The mixture is suspended in an appropriate aqueous medium, lyophilised and sterilized by gamma irradiation.

The purity measured on the obtained pharmaceutical biodegradable composition is 98.8% and the burst evaluated in vitro (in a phosphate buffer pH 7.4) over a 6 hours period is 45.0%.

In this example, the obtained pharmaceutical formulation is tested in vivo and the animal model is the rat. The formulation as described above is suspended in water for injection and is administered at a concentration dose of 18 mg/kg to 6 rats.

The LHRH agonist triptorelin of said pharmaceutical biodegradable composition is released in an important immediate amount within hours following injection and then shows a constant and significant release over a long period of at least 168 days, i.e. 6 months (see FIG. 2).

### Example 4

A formulation of microspheres of triptorelin pamoate and PLGA 95/5 having an inherent viscosity of 0.18 dl/g is prepared as follows:

Aqueous phase is prepared by mixing, under magnetic stirring, at a temperature of 40° C., 800 g of polyvinyl alcohol and 40 L of purified water. In parallel, the organic phase is prepared by total dissolution of 80 g of PLGA 95/5 in 334 g of isopropyl acetate under magnetic stirring.

20 g of triptorelin pamoate are suspended in 100 g of isopropyl acetate and placed under magnetic stirring. This solution is incorporated to the organic phase previously prepared. The organic phase is then introduced in a homogenisation chamber simultaneously with the said aqueous phase. Both phases are mixed in order to obtain an emulsion and the extraction of the solvent from the organic phase and to isolate a suspension of microspheres.

Finally the formulation of microspheres is recovered by filtration and dried by lyophilization.

The microspheres have a size distribution defined as follows:
D (v,0.1)=17.8 micrometers
D (v,0.5)=37.1 micrometers
D (v,0.9)=74.8 micrometers

This formulation of microspheres is suspended in an appropriate aqueous medium, lyophilised and sterilized by gamma irradiation.

The purity measured on the obtained pharmaceutical biodegradable composition is 99.2% and the burst evaluated in vitro (in a phosphate buffer pH 7.4) over a 6 hours period is 10.9%.

In this example, the obtained pharmaceutical formulation is tested in vivo and the animal model is the rat. The formulation as described above is suspended in water for injection and is administered at a concentration dose of 18 mg/kg to 6 rats.

The LHRH agonist triptorelin of said pharmaceutical biodegradable composition is released in an important immediate amount within hours following injection and then shows a constant and significant release over a long period of at least 168 days, i.e. 6 months (see FIG. 3).

### Example 5

In order to increase patients' compliance and convenience the inventors also developed a formulation as defined in previous example 3 which allows one injection every 6 Months (24 Weeks). The study discussed in this example investigated the efficacy and safety of this formulation after 2 consecutive intramuscular injections of triptorelin pamoate 22.5 mg in 120 patients with advanced prostate cancer. Four-weekly testosterone assessments were performed over 48 weeks.

Serum testosterone concentrations fell to castrate levels (≤1.735 nmol/L) in 97.5% of the patients on D29, and 93% of the patients maintained castration from Week 8 to 48. Five out of 8 patients who escaped castration had only an isolated testosterone breakthrough without rising PSA (Prostate Specific Antigen), indicating maintained efficacy. Only one of these isolated breakthroughs was a true “acute-on-chronic” phenomenon after the second injection.

The median relative decreases in PSA from baseline were 96.9% at Week 24, and 96.4% at Week 48, when 80.9% of patients had a normal PSA.

The type and incidence of AEs (Adverse Events) were comparable with those observed with the registered triptorelin formulations. As with other GnRH agonists, the most frequent drug related AEs were hot flushes (71.7% of patients). The study drug was very well tolerated locally.

The study discussed above shows that Triptorelin 6-Month formulation is efficacious and safe in inducing chemical castration in patients with advanced prostate cancer. This new convenient formulation requires only 1 injection every 24 weeks, and shows comparable efficacy and safety with the marketed 1- and 3-Month formulations.

1-15. (canceled)

16. A pharmaceutical composition comprising microparticles comprising 22.5 mg triptorelin in the form of triptorelin pamoate, wherein the microparticles are made of poly(D,L lactide-co-glycolide) (PLGA), wherein the PLGA comprises at least 75% lactide, and wherein the triptorelin pamoate is released from the pharmaceutical composition in an immediate amount within hours following injection into a patient and then constantly released over a period of at least 168 days, wherein the composition comprises two groups of microparticles, wherein the two groups of microparticles are a first group of microgranules and a second group of microgranules.

17. The pharmaceutical composition of claim 16, wherein the first group of microgranules and the second group of microgranules are mixed to have a 50:50 dose ratio of triptorelin.

18. The pharmaceutical composition of claim 16, wherein the pharmaceutical composition is administered to the patient once about every six months.

19. The pharmaceutical composition of claim 16, wherein the PLGA in the first group of microgranules comprises at least 85% lactide.

20. A method of treating prostate cancer in a patient in need thereof comprising administering to the patient a pharmaceutical composition comprising microparticles comprising 22.5 mg triptorelin in the form of triptorelin pamoate, wherein the microparticles are made of poly(D,L lactide-c0-glycolide) (PGLA), wherein the PLGA comprises at least 75% lactide, and wherein the triptorelin pamoate is released from the pharmaceutical composition in an immediate amount within hours following injection and then constantly released over a period of at least 168 days, wherein the composition comprises two groups of microparticles, wherein the two groups of microparticles are a first group of microgranules and a second group of microgranules.

21. The method of claim 20, wherein the composition is administered once every 6 months to the patient.

22. The method of claim 20, wherein the first group of microgranules is a first formulation of microgranules comprising approximately 20% (w/w) of triptorelin pamoate mixed with approximately 80% (w/w) PLGA, wherein the PLGA in the first formulation contains approximately 85% lactide and 15% glycolide and the second group of microgranules is a second formulation of microgranules comprising approximately 12% (w/w) triptorelin pamoate mixed with approximately 88% (w/w) poly(D,L lactide-co-glycolide) (PLGA), wherein the PLGA in the second formulation contains approximately 75% lactide and 25% glycolide.

23. The method of claim 20, wherein the first group of microgranules and the second group of microgranules are mixed to have a 50:50 dose ratio of triptorelin.

24. The method of claim 20, wherein the pharmaceutical composition induces chemical castration in the patient over a six-month period of time.

25. The method of claim 20, wherein the patient has advanced prostate cancer.

26. The method of claim 20, wherein the PLGA in the first group of microgranules contains at least 85% lactide.
